(19) United States
(12) Patent Application Publication
(10) Pub. No.: US 2004/0072877 A1
(43) Pub. Date: Apr. 15, 2004

(54) Method for increasing insulin sensitivity and for treating and preventing type 2 diabetes

(76) Inventors: James M. Ntambi, Madison, WI (US); Alan D. Attie, Madison, WI (US); Makoto Miyazaki, Madison, WI (US)

Correspondence Address: Bennett J. Berson, Quarles & Brady LLP, P O Box 2113, Madison, WI, 53701-2113, US

(21) Appl. No.: 10/620,404

(22) Filed: Jul. 16, 2003

Related U.S. Application Data
(60) Provisional application No. 60/398,471, filed on Jul. 25, 2002.

(51) Int. Cl. A61K 48/00; A61K 31/4439; A61K 31/426; A61K 31/202

(52) U.S. Cl. 514/342; 514/365; 514/44; 514/560

(57) ABSTRACT

It is disclosed here that insulin sensitivity in a human or non-human animal can be increased by reducing stearoyl-CoA desaturase-1 (SCD1) activity in the animal. This provides a new tool for treating and preventing type 2 diabetes. Also disclosed are methods for identifying agents that can increase insulin sensitivity in a human or non-human animal through determining the agents' effects on SCD1 activity.

### CROSS-REFERENCE TO RELATED APPLICATIONS

This application claims priority to U.S. provisional application Serial No. 60/398,471, filed on Jul. 25, 2002, which is incorporated by reference in its entirety.

### STATEMENT REGARDING FEDERALLY SPONSORED RESEARCH OR DEVELOPMENT

This invention was made with United States government support awarded by the following agency: USDA 01-CRHF-0-6055. The United States has certain rights in this invention.

### BACKGROUND OF THE INVENTION

Over 90% of diabetes patients have type 2 diabetes. The American Diabetes Association reports that there are 12 million Americans with type 2 diabetes and another 7 million potential candidates. An annual expenditure of $100 billion is attributed to the disease. It is the third leading cause of death at 62,000 each year. Prolonged untreated diabetes leads to heart diseases, stroke, kidney disease, blindness, and loss of limbs from advanced peripheral vascular disease.

Type 2 diabetes is also called non-insulin dependent diabetes mellitus (NIDDM) because unlike type 1 diabetes wherein patients lose the ability to produce insulin in the pancreas, type 2 diabetes patients do produce insulin but their bodies do not respond to insulin signaling to lower the blood glucose level. The lack of response is due at least in part to the impairment of glucose transport in insulin sensitive tissues (Cline, G. W. et al. (1999) N. Engl. J. Med. 341, 240-246; Garvey, W. T. et al. (1988) J. Clin. Invest. 81, 1528-1536). Skeletal muscle represents the most important tissue for the maintenance of a balanced postprandial glucose homeostasis; about 80% of insulin-stimulated glucose uptake is accounted for by muscle tissue (Baron, A. D. et al. (1988) Am. J. Physiol. 255, E769-E774). In skeletal muscle and other insulin sensitive tissues, insulin increases glucose transport into cells by stimulating the translocation of the glucose transporter isoform 4 (GLUT4) from an intracellular pool to the plasma membrane (Hirshman, M. F. et al. (1990) J. Biol. Chem. 265, 987-991; Cushman, S. W., and Wardzala, L. J. (1980) J. Biol. Chem. 255, 4758-4762). The intracellular signaling pathway by which insulin mediates glucose transport involves signal transduction through the insulin receptor (IR), whereby insulin binding to the α subunit of the insulin receptor derepresses the kinase activity in the β-subunit followed by tyrosine autophosphorylation of the α-subunit and a conformational change in the receptor structure that further increases tyrosine kinase activity towards insulin receptor substrates (IRSs) (Withers, D. J. and White, M. (2000) Endocrinology. 141, 1917-1921). IRS tyrosine phosphorylation leads to activation of phosphatidylinositol 3-kinase (PI 3-kinase) and Akt/PKB (Holman, G. D., and Kasuga, M. (1997) Diabetologia. 40, 991-1003; Kohn, A. D. et al. (1995) EMBO J. 14,4288-4295) which are key signaling transducers in insulin-mediated GLUT4 translocation, glucose uptake and glycogen synthesis (Kohn, A. D. et al. (1996) J. Biol. Chem. 271, 3137-8; Tanti, J. F. (1997) Endocrinology 138, 200-210; Thompson, A. L. et al. (200) Am. J. Physiol. 279, E577-E584). Protein tyrosine phosphatase-1B (PTP-1B) that has been implicated in the negative regulation of insulin signaling dephosphoryalates the activated insulin receptor thereby attenuating the insulin response. PTP-1B−/− mice have sustained insulin response because the insulin receptor remains phosphorylated and therefore activated longer than in the PTP-1B+/+ mice (Elchebly, M. et al. (1999) Science 283, 1544-1548).

Obesity has been identified as an independent risk factor for the development of type 2 diabetes. More than 80% of type 2 diabetic patients are obese. For patients who have developed diabetes, cardiovascular diseases caused by atherosclerosis (thickening of large blood vessels) account for approximately 25% of the deaths. The fatty acid profile in diabetic patients is closely monitored. One of the lipogenic enzymes, stearoyl-CoA desaturase (SCD), is a key enzyme in the biosynthesis of compounds, such as phospholipids, triglyceride and cholesterol esters, that are related to fat metabolism and atherosclerosis. However, SCD has not been implicated in the treatment of type 2 diabetes.

SCD belongs to the enzyme family of acyl desaturases, which catalyze the formation of double bonds in fatty acids derived from either dietary sources or de novo synthesis in the liver and other tissues. Mammals possess four desaturases of differing chain length specificity that catalyze the addition of double bonds at the delta-9, delta-6, delta-5 and delta-4 positions. SCD is a microsomal enzyme that catalyzes the synthesis of monounsaturated fatty acids by introducing the cis double bond in the delta-9 position of saturated fatty acyl-CoAs. The preferred desaturation substrates of SCD are palmitoyl-CoA and stearoyl-CoA, which are converted to palmitoleoyl-CoA (16:1) and oleoyl-CoA (18:1), respectively (Enoch, H. G., and Strittmatter, P. (1978) Biochemistry. 17, 4927-4932). These monounsaturated fatty acids are used as substrates for the synthesis of triglycerides, wax esters, cholesteryl esters and membrane phospholipids (Miyazaki, M. et al. (2000) J. Biol. Chem 275, 30132-30138; Miyazaki, M. et al. (2001) J. Lipid Res. 42,1018-1024; Miyazaki, M. et al. (2001) J. Nutr. 131, 2260-2268).

A single human and four mouse SCD isoforms (SCD1, SCD2, SCD3 and SCD4) have been characterized (Ntambi, J. M. et al. (1988) J. Biol. Chem. 263, 17291-17300; Kaestner, K. H. et al. (1989) J. Biol. Chem. 264, 14755-14761; Bene, H., Lasky, D., and Ntambi, J. M. (2001) Biochem. Biophys. Res. Commun. 284, 1194-1198; Zhang, L. et al. (1999). Biochem. J. 340, 255-264). New insights into the physiological role of the SCD1 gene and its endogenous products have come from recent studies of the asebia mouse strains (ab^{j} and ab^{2j}) that have a naturally-occurring mutation in SCD1 gene (Zhang, L. et al. (1999). Biochem. J. 340, 255-264; Zheng, Y. et al. (1999) Nature Genet. 23, 268-270; Zheng, Y. et al. (2001) Genomics. 71, 182-191) as well as a laboratory mouse model with a targeted disruption in the SCD1 gene (SCD1−/−) (4). SCD1−/− mice are found to be deficient in tissue triglycerides, cholesterol esters, wax esters and 1-alkyl-2, 3-diacylglycerol (Miyazaki, M. et al. (2000) J. Biol. Chem 275, 30132-30138; Miyazaki, M. et al. (2001) J. Lipid Res. 42,1018-1024; Miyazaki, M. et al. (2001) J. Nutr. 131, 2260-2268).

### SUMMARY OF THE INVENTION

In one aspect, the present invention relates to a method for increasing insulin sensitivity in a human or non-human subject. The method includes the step of reducing stearoyl-CoA desaturase 1 (SCD1) activity in the human or non-human subject sufficiently to increase insulin sensitivity. This can be accomplished by reducing the amount of SCD1 protein, by inhibiting the SCD1 enzymatic activity, or both. Type 2 diabetes can be treated or prevented by practicing this method.

In another aspect, the present invention relates to a method for identifying an agent that can increase insulin sensitivity in a human or non-human subject. In one embodiment, the method includes the steps of providing a preparation that contains SCD 1 activity, contacting the preparation with a test agent, measuring the SCD 1 activity of the preparation, and comparing the activity to that of a control preparation that is not exposed to the test agent. A lower than control activity indicates that the agent can increase insulin sensitivity in a human or non-human subject. In another embodiment, the method includes the steps of administering a test agent to the human or non-human subject and determining the effect of the agent on the SCD 1 activity. If the SCD 1 activity is reduced, it indicates that the agent can increase insulin sensitivity in a human or non-human subject.

### BRIEF DESCRIPTION OF THE DRAWINGS

FIG. 1 shows insulin receptor, IGF-1 receptor, IRS-1 and IRS-2 phosphorylation status and protein levels in muscle of SCD1−/− and SCD1+/+ mice. Gastrocnemius muscles from 3 SCD+/+ and 3 SCD1−/− mice were pooled and homogenized as described in Example 1. Equal amount of muscle proteins obtained were immunoprecipitated with β-subunit of insulin receptor (IR), IRS-1, IRS-2, and β-subunit of IGF-1 receptor antibodies, separated by SDS-PAGE, and subjected to immunoblotting analysis with αPY antibodies. Each experiment was repeated three times. Intensity of the bands was quantified by densitometry. Net intensity of the bands was normalized for the total protein content of the samples. Nitrocellulose membranes were stripped and reprobed with IR, IRS-1, IRS-2 and IGF-1R antibodies to ensure equal loading of the proteins. Representative immunoblot along with combined densitometric analysis are shown. (A) Insulin receptor and IGF-1 receptor phosphorylation and protein levels. IR-P, IR tyrosine phosphorylation; IGF-1R-P, IGF-1R tyrosine phosphorylation. (B) IRS-1 phosphorylation (IRS-1-P) and protein (IRS-1-protein). (C) IRS-2 phosphorylation (IRS-2-P) and protein (IRS-2-protein). Tyrosine phosphorylation of IR, IRS-1 and IRS-2 was expressed as fold change. Data are means ±SD, ***P<0.0005, **P<0.005, *P<0.01 vs. controls.

FIG. 2 shows association of insulin receptor substrates (IRS-1 and IRS-2) with αp85 subunit of PI 3-kinase and αp85 abundance in muscle. Gastrocnemius muscles from 3 SCD+/+ and 3 SCD1−/− mice were pooled and homogenized as described in Example 1. Equal amount of muscle proteins obtained were immunoprecipitated (IP) with IRS-1 and IRS-2 antibodies separated by SDS-PAGE, and subjected to immunoblotting analysis with αp85 subunit of PI3kinase. For the measurement of αp85 protein level, equal amount of protein was separated by SDS-PAGE and immunoblotted with αp85 antibody. Each experiment was repeated three times. Intensity of the bands was quantified by densitometry. Net intensity of the bands was normalized for the total protein content of the samples. Representative immunoblot along with combined densitometric analysis are shown. (A) Association of IRS-1 with αp85. (B) Association of IRS-2 with αp85. (C) p85 protein level. Data are means ±SD. *P<0.05, **P<0.01 vs. controls.

FIG. 3 shows that mRNA, protein level and activity of PTP-IB are reduced in the SCD1−/−mice. (A) PTP-1B mRNA levels. Total RNA was isolated from pooled gastrocnemius muscle of 3 SCD1−/and 3 SCD1+/+ mice and were subjected to RT-PCR using cyclophilin as a control. Each experiment was repeated three times. Data is expressed as percent of control. *P<0.001 vs controls. (B) Representative immunoblot of PTP-1B and LAR protein levels along with combined densitometric analysis of the PTP-1B levels are shown. Homogenates from muscle of SCD1−/− and SCD1+/+ mice were centrifuged and the supernatants collected. Equal amount of muscle proteins were separated by SDS-PAGE and subjected to Immunoblotting analysis with anti PTP-IB antibody. Protein was quantified by scanning densitometry and expressed as percent of control. Experiment was repeated three times. Data are means ±SD, *P<0.001 vs controls (SCD1+/+). Nitrocellulose membrane was stripped and reprobed with GAPDH antibody to ensure equal loading of the protein. (C) PTP-1B activity. Muscle tissues isolated from 3 SCD1−/− and 3 SCD1+/+ mice were homogenized and supernatant was collected for immunoprecipitation with anti PTP-1B antibody. PTP-1B immunocomplexes were used to measure phosphatase activity. Activity was expressed as percent of control. Data are shown as means ±SD, *P<0.001 vs controls.

FIG. 4 shows that Akt/PKB phosphorylation is increased in muscle of SCD1−/− mice. Muscle samples from 3 SCD1+/+ and 3 SCD1−/− mice were homogenized as described in Example 1. Representative immunoblots are shown (A) along with denstometric quantification (B, and C). Equal amount of protein was separated by SDS-PAGE and immunoblotted with polyclonal antibodies against phospho-Ser 473-Akt or phospho-Thr 308-Akt. Net intensity of the bands was normalized for the total protein content of the samples. Experiment was repeated three times. All data are shown as means ±SD, *P<0.005 vs. controls.

FIG. 5 shows expression and quantification of GLUT4 and glucose uptake in muscle of SCD1−/− and SCD1+/+ mice. (A) Representative immunoblot of GLUT4 protein expression along with combined densitometric analysis. Muscle from 3 SCD1+/+ and 3 SCD1−/− mice were pooled. Plasma membranes were prepared as described in Example 1. Equal amount of protein was separated by SDS-PAGE and immunoblotted with GLUT4 antibody. Experiment was repeated three times. Data are shown as means ±SD. *P<0.05 vs controls. Nitrocellulose membrane was stripped and reprobed with GAPDH antibody to ensure equal loading of the protein. (B) Glucose uptake measured in vivo in soleus and gastrocnemius muscles. Mice were anesthetized and 0.2 μCi of 2-deoxy-D-[1-¹⁴C] glucose and 0.8 μCi of [1−³H] mannitol per 20 g body wt were administered into the tail vein. The muscles were taken 25 min after. Data are shown as means ±SD. **P<0.01; n=5 mice/group. (C) Basal and insulin-stimulated glucose uptake in isolated soleus muscle from control and SCD1−/− mice. The soleus muscles were preincubated in Krebs-Ringer bicarbonate buffer with 0.1 m-unit of insulin/ml [insulin (+)] or without insulin [insulin (−)] for 2h. The muscles were then transferred to fresh identical medium supplemented with 1 mM 2-deoxy-D-[1-¹⁴C] glucose and 0.5 mM [1-³H] mannitol for an additional 15 min to measure glucose uptake. The 2-deoxyglucose uptake was calculated as the difference between the total muscle radioactivity and the radioactivity of the muscle extracellular space measured using [1-³H] mannitol. Data are means ±SD for 5 mice/group. ***P<0.0001 vs. controls.

FIG. 6 shows enzyme activities in muscle of SCD1−/− and SCD1+/+ mice. (A) Glycogen synthase activities in muscle. Glycogen synthase activities were measured in both the presence (total) and absence (active) of G6P. (B) Glycogen phosphorylase activities. Glycogen phosphorylase activities were measured in both the presence (total) and absence (active) of AMP. Data are means ±SD for 3 mice/group. *P<0.05 vs. controls.

FIG. 7 shows muscle glycogen content. Values are means ±SD for 3 mice/group. *P <0.001.

FIG. 8 body weight of male and female wild-type and SCD1−/− mice fed a chow or high-fat diet.

FIG. 9 shows reduced body fat mass in SCD−/− mice. (A) Abdominal view of the fat pad under the skin in 23-week-old male wild-type and SCD1−/− mice. (B) Epididymal fat pads and liver isolated from the wild-type and SCD1−/− mice on a chow diet. (C) Epididymal fat pads and liver isolated from the wild-type and SCD1−/− mice on a high-fat diet. (D) Fat pad weights from mice fed chow and high-fat diets.

FIG. 10 shows increased oxygen consumption in SCD1−/− mice. (A) Metabolic rate and oxygen consumption of male mice on a chow diet. (B) Gender-adjusted, normalized total oxygen consumption over a 23-h period. Error bars denote SE.

FIG. 11 shows increased expression of genes involved in fatty acid oxidation in SCD1−/− mice. (A) Expression levels of lipid oxidation (left) and lipid synthesis (right) genes between wild-type and SCD1−/− mice. (B) Quantitative reverse-transcription-PCR of FIAF and FAS gene expression, relative to wild-type mice. 18S RNA was used as a normalization control. (C) Northern blot analysis of lipid oxidation genes and lipid synthesis genes (SREBP-1, FAS, and GPAT) in the wild-type and SCD1−/− mice.

FIG. 12 shows plasma glucose levels during the glucose tolerance test of male and female wild-type and SCD1−/− mice.

### DETAILED DESCRIPTION OF THE INVENTION

1. Increasing Insulin Sensitivity

The present invention discloses that insulin sensitivity in a human or non-human animal can be increased by reducing stearoyl-CoA desaturase-1 (SCD 1) activity in the animal. For the purpose of the present invention, increased insulin sensitivity means a higher rate of cellular glucose uptake and a greater reduction in blood glucose level in response to the same amount of insulin or increase in insulin level in a human or non-human animal. Therefore, type 2 diabetes can be treated or prevented by reducing the SCD 1 activity in the patients. The term “prevent” used broadly here to include delaying of the onset of a disease, reducing in the severity of a disease at the onset, or completely preventing the development of a disease. To simplify the language of the disclosure, the terms “animal” and “subject” will be used here to refer both to humans and non-human animals.

The increase in insulin sensitivity by reducing SCD 1 activity is demonstrated in the examples below. In SCD1 knockout mice (SCD1−/−), even though the insulin level was decreased in comparison to the wild-type mice, the activity of the insulin signaling pathway was increased. The insulin pathway starts with the binding of insulin to its receptor, which triggers a cascade of signal transduction events, and ends with an increase in cellular uptake of glucose and a reduction in blood glucose level. For all the components of the insulin pathway that were measured in the examples below, increased activities were detected. Although the effect of higher insulin sensitivity was demonstrated by genetic manipulation, genetic manipulation is not required for the effect to occur. What is necessary is for the level of SCD 1 activity in a human or non-human subject be lowered. This can be done through genetic manipulation or through the use of other modulators of SCD1 activity.

The effect described here is effective for any of the various SCDs in various animal species that correspond to the mouse SCD1. A skilled artisan is familiar with these corresponding SCDs. For example, in humans, a single SCD gene has been identified and it corresponds to the mouse SCD1 gene. To simplify the language of the disclosure, the term SCD1 is used generally for all SCDs that correspond to mouse SCD 1. The SCD1s cloned from different mammalian species show a high degree of homology. For example, the human SCD1 protein (GenBank Accession No. O00767) and the mouse SCD1 protein (GenBank Accession No. P13516) show about 87% sequence identity at the amino acid level. From the perspective of desaturating a saturated fatty acid C_{18:0} to C_{18:1} at the delta-9 position, the activity of SCD1 in different animals are conserved. It is expected that reducing the activity of a SCD1 can be used as a method for increasing insulin sensitivity in an animal in general. The animals include but are not limited to mammals. The mammals include but are not limited to human beings, primates, bovines, canines, porcines, ovines, caprines, felines and rodents.

Any agent that is known to a skilled artisan to reduce SCD1 activity but which does not significantly cross-react with other desaturases can be used in the present invention. New agents identified to be able to reduce SCD1 activity can also be used. Agents can be administered orally, as a food supplement or adjuvant, or by any other effective means which has the effect of reducing SCD1 activity.

While it is envisaged that any suitable mechanism for reducing SCD1 activity can be used, three specific examples of reduction classes are envisioned. One class includes lowering SCD1 protein level. A second class includes the inhibition of SCD1 enzymatic activity. The third class includes interfering with the proteins essential to the desaturase system, such as cytochrome b₅, NADH (P)-cytochrome b₅ reductase, and terminal cyanide-sensitive desaturase.

Many strategies are available to lower SCD1 protein level. For example, one can increase the degradation rate of the enzyme or inhibit rate of synthesis of the enzyme. The synthesis of the enzyme can be inhibited at transcriptional level or translational level by known genetic techniques. Since SCD1 is regulated by several known transcription factors (e.g. PPAR-γ, SREBP), any agent that affects the activity of such transcription factors can be used to alter the expression of the SCD1 gene at the transcriptional level. One group of such agents includes thiazoladine compounds which are known to activate PPAR-γ and inhibit SCD1 transcription. These compounds include Pioglitazone, Ciglitazone, Englitazone, Troglitazone, and BRL49653. Another agent is leptin, which has been shown to inhibit SCD1 expression (Cohen, P. et al., Science. 297: 240-243, 2002, incorporated herein by reference in its entirety). Other transcription inhibitory agents may include polyunsaturated fatty acids, such as linoleic acid, arachidonic acid and dodecahexaenoic acid.

One method to block SCD1 synthesis at the translational level is to use an antisense oligonucleotide (DNA or RNA) having a sequence complementary to at least part of a SCD1 mRNA sequence. One of ordinary skill in the art knows how to make and use an antisense oligonucleotide to block the synthesis of a protein (Agarwal, S. (1996) Antisense Therapeutics. Totowa, N.J., Humana Press, Inc.). An example of the antisense method for the present invention is to use 20-25 mer antisense oligonucleotides directed against 5′ end of a SCD1 mRNA with phosphorothioate derivatives on the last three base pairs on the 3′ end and the 5′ end to enhance the half life and stability of the oligonucleotides. A useful strategy is to design several oligonucleotides with a sequence that extends 2-5 basepairs beyond the 5′ start site of transcription.

An antisense oligonucleotide used for increasing insulin sensitivity can be administered intravenously into an animal. A carrier for an antisense oligonucleotide can be used. An example of a suitable carrier is cationic liposomes. For example, an oligonucleotide can be mixed with cationic liposomes prepared by mixing 1-alpha dioleylphatidylcelthanolamine with dimethldioctadecylammonium bromide in a ratio of 5:2 in 1 ml of chloroform. The solvent will be evaporated and the lipids resuspended by sonication in 10 ml of saline.

Another way to use an antisense oligonucleotide is to engineer it into a vector so that the vector can produce an antisense cRNA that blocks the translation of the mRNAs encoding for SCD1.

Several agents are known to inhibit SCD1 activity. For example, certain conjugated linoleic acid isomers are effective inhibitors of SCD1 activity. Specifically, cis-12, trans-10 conjugated linoleic acid and various derivatives thereof are known to effectively inhibit SCD1 enzymatic activity and reduce the abundance of SCD1 mRNA (Park, Y. et al., Biochim Biophys Acta. 1486(2-3):285-292, 2000, incorporated herein by reference in its entirety). Cyclopropenoid fatty acids, such as those found in stercula and cotton seeds, are also known to inhibit SCD activity. For example, sterculic acid (8-(2-octyl-cyclopropenyl)octanoic acid) and malvalic acid (7-(2-octyl-cyclopropenyl)heptanoic acid) are C18 and C16 derivatives of sterculoyl- and malvaloyl-fatty acids, respectively, having cyclopropene rings at their delta-9 position. These agents as well as the active derivatives and analogous thereof inhibit SCD1 activity by inhibiting delta-9 desaturation (U.S. Pat. No. 4,910,224, incorporated herein by reference in its entirety). Other agents include thia-fatty acids, such as 9-thiastearic acid (also called 8-nonylthiooctanoic acid) and other fatty acids with a sulfoxy moiety.

Although the conjugated linoleic acids, cyclopropene fatty acids (malvalic acid and sterculic acid) and thia-fatty acids can inhibit SCD1 activity, the inhibition is not specific in that they inhibit other desaturases as well, in particular the delta-5 and delta-6 desaturases by the cyclopropene fatty acids. In addition, the inhibition of SCD1 activity by these acids may require very high dosage. Thus, these compounds themselves are not preferred agents for increasing insulin sensitivities in animals. However, they can be useful for establishing control for the screening assays of the invention. Preferred SCD1 inhibitors of the invention have no significant or substantial impact on unrelated classes of proteins. In some cases, assays specific for the other proteins, such as delta-5 and delta-6 activity, will also need to be tested to ensure that the identified compounds of the invention do not demonstrate significant or substantial cross inhibition.

The known non-specific inhibitors of SCD1 can also be useful in rational design of a therapeutic agent suitable for inhibition of SCD1. The conjugated linoleic acids, cyclopropene fatty acids and thia-fatty acids have various substitutions between carbons #9 and #10, require conjugation to CoA to be effective, and are probably situated in a relatively hydrophobic active site of SCD1. This information combined with the known X-ray co-ordinates for the active site for plant (soluble) SCD can assist the “in silico” process of rational drug design for therapeutically acceptable inhibitors specific for SCD1.

Besides the SCD1 enzyme inhibitors described above, a SCD1 monoclonal or polyclonal antibody, or an SCD1-binding fragment thereof, can also be used as enzyme inhibitors for the purpose of this invention. In one embodiment, the antibody is isolated, i.e., an antibody free of any other antibodies. Generally, it has been shown that an antibody can block the function of a target protein when administered into the body of an animal. Dahly, A. J., FASEB J. 14:A133, 2000; Dahly, A. J., J. Am. Soc. Nephrology 11:332A, 2000. Thus, a SCD1 antibody can be used to increase insulin sensitivity in a human or non-human animal. For example, about 0.01 mg to about 100 mg, preferably about 0.1 mg to about 10 mg, and most preferably about 0.2 mg to about 1.0 mg of humanized SCD1 antibodies can be administered to a human being. The half life of these antibodies in a human being can be as long as 2-3 weeks. For the SCD1s whose DNA and protein amino acid sequences are published and available, one of ordinary skill in the art knows how to make monoclonal or polyclonal antibodies against them (Harlow, et al. 1988. Antibodies: A Laboratory Manual; Cold Spring Harbor, N.Y., Cold Spring Harbor Laboratory).

An agent that interferes with a protein essential to the desaturase system can also be used to inhibit SCD1 activity. The desaturase system has three major proteins: cytochrome b₅, NADH (P)-cytochrome b₅ reductase, and terminal cyanide-sensitive desaturase. Terminal cyanide-sensitive desaturase is the product of the SCD gene. SCD activity depends upon the formation of a stable complex between the three aforementioned components. Thus, any agent that interferes with the formation of this complex or any agent that interferes with the proper function of any of the three components of the complex would effectively inhibit SCD1 activity.

II. Screening Assays

Since the present invention is based on reducing SCD 1 activity levels, screening assays employing the SCD1 gene and/or protein for identifying agents that inhibit SCD1 expression or enzymatic activity will identify candidate drugs for increasing insulin sensitivity in an animal.

1. “SCD1 Biological Activity”

“SCD1 biological activity” used herein, especially relating to screening assays, is interpreted broadly and contemplates all directly or indirectly measurable and identifiable biological activities of the SCD1 gene and protein. Relating to the purified SCD 1 protein, SCD1 biological activity includes, but is not limited to, all those biological processes, interactions, binding behavior, binding-activity relationships, pKa, pD, enzyme kinetics, stability, and functional assessments of the protein. Relating to SCD1 biological activity in cell fractions, reconstituted cell fractions or whole cells, these activities include, but are not limited to the rate at which the SCD introduces a cis-double bond in its substrates palmitoyl-CoA (16:0) and stearoyl-CoA (18:0), which are converted to palmitoleoyl-CoA (16:1) and oleoyl-CoA (18:1), respectively, and all measurable consequences of this effect, such as triglyceride, cholesterol or other lipid synthesis, membrane composition and behavior, cell growth, development or behavior, and other direct or indirect effects of SCD1 activity. Relating to SCD 1 genes and transcription, SCD1 biological activity includes the rate, scale or scope of transcription of genomic DNA to generate RNA, the effect of regulatory proteins on such transcription, the effect of modulators of such regulatory proteins on such transcription, and the stability and behavior of mRNA transcripts, post-transcription processing, mRNA amounts and turnover, and all measurements of translation of the mRNA into polypeptide sequences. Relating to SCD1 biological activity in organisms, this includes but is not limited to biological activities which are identified by their absence or deficiency in disease processes or disorders caused by aberrant SCD1 biological activity in those organisms. Broadly speaking, SCD1 biological activity can be determined by all these and other means for analyzing biological properties of proteins and genes that are known in the art.

2. Design and Development of SCD Screening Assays

The present disclosure facilitates the development of screening assays that may be cell based, cell extract (e.g. microsomal assays) or cell free (e.g. transcriptional) assays, and assays of substantially purified protein activity. Such assays are typically radioactivity or fluorescence based (e.g. fluorescence polarization or fluorescence resonance energy transfer (FRET)), or they may measure cell behavior (viability, growth, activity, shape, membrane fluidity, temperature sensitivity etc). Alternatively, screening may employ multicellular organisms, including genetically modified organisms such as knock-out or knock-in mice, or naturally occurring genetic variants. Screening assays may be manual or low throughput assays, or they may be high throughput screens which are mechanically/robotically enhanced.

The aforementioned processes afford the basis for screening processes, including high throughput screening processes, for determining the efficacy of potential agents for increasing insulin sensitivity.

The assays disclosed herein essentially require the measurement, directly or indirectly, of an SCD1 biological activity. Those skilled in the art can develop such assays based on well known models, and many potential assays exist. For measuring whole cell activity of SCD1 directly, methods that can be used to quantitatively measure SCD activity include for example, measuring thin layer chromatographs of SCD reaction products over time. This method and other methods suitable for measuring SCD activity are well known (Henderson Henderson “RJ, et al. 1992. Lipid Analysis: A Practical Approach. Hamilton S. Eds. New York and Tokyo, Oxford University Press. pp 65-111). Gas chromatography is also useful to distinguish monounsaturates from saturates, for example oleate (18:1) and stearate (18:0) can be distinguished using this method. These techniques can be used to determine if a test compound has influenced the biological activity of SCD1, or the rate at which the SCD introduces a cis-double bond in its substrate palmitate (16:0) or stearate (18:0) to produce palmitolyeoyl-CoA (16:1) or oleyoyl-CoA (18:1), respectively.

In one embodiment of an SCD1 activity assay, the assay employs a microsomal assay having a measurable SCD1 biological activity. A suitable assay may be taken by modifying and scaling up the rat liver microsomal assay essentially as described by Shimomura et al. (Shimomura, I., Shimano, H., Kom, B. S., Bashmakov, Y., and Horton, J. D. (1998)). Tissues are homogenized in 10 vol. of buffer A (0.1 M potassium buffer, pH 7.4). The microsomal membrane fractions (100,000×g pellet) are isolated by sequential centrifugation. Reactions are performed at 37° C. for 5 min with 100 μg of protein homogenate and 60 μM of [1 −¹⁴C]-stearoul-CoA (60,000 dpm), 2 mM of NADH, 0.1 M of Tris/HCI buffer (pH 7.2). After the reaction, fatty acids are extracted and then methylated with 10% acetic chloride/methanol. Saturated fatty acid and monounsaturated fatty acid methyl esters are separated by 10% AgNO₃-impregnated TLC using hexane/diethyl ether (9:1) as developing solution. The plates are sprayed with 0.2% 2′, 7′-dichlorofluorescein in 95% ethanol and the lipids are identified under UV light. The fractions are scraped off the plate, and the radioactivity is measured using a liquid scintillation counter.

Specific embodiments of such SCD1 biological activity assay take advantage of the fact that the SCD reaction produces, in addition to the monounsaturated fatty acyl-CoA product, H₂O. If ³H is introduced into the C-9 and C-10 positions of the fatty-acyl-CoA substrate, then some of the radioactive protons from this reaction will end up in water. Thus, the measurement of the activity would involve the measurement of radioactive water. In order to separate the labeled water from the stearate, investigators may employ substrates such as charcoal, hydrophobic beads, or just plain old-fashioned solvents in acid pH.

In another embodiment, screening assays measure SCD1 biological activity indirectly. Standard high-throughput screening assays center on ligand-receptor assays. These may be fluorescence based or luminescence based or radiolabel detection. Enzyme immunoassays can be run on a wide variety of formats for identifying compounds that interact with SCD1 proteins. These assays may employ prompt fluorescence or time-resolved fluorescence immunoassays which are well known. ³²P labeled ATP is typically used for protein kinase assays. Phosphorylated products may be separated for counting by a variety of methods. Scintillation proximity assay technology is an enhanced method of radiolabel assay. All these types of assays are particularly appropriate for assays of compounds that interact with purified or semi-purified SCD1 protein.

In yet another embodiment, the assay makes use of ³H-stearoyl CoA (with the ³H on the 9 and 10 carbon atoms), the substrate for SCD1. Desaturation by SCD1 produces oleoyl CoA and ³H -water molecules. The reaction is run at room temperature, quenched with acid and then activated charcoal is used to separate unreacted substrate from the radioactive water product. The charcoal is sedimented and amount of radioactivity in the supernatant is determined by liquid scintillation counting. This assay is specific for SCD1-dependent desaturation as judged by the difference seen when comparing the activity in wild type and SCD1-knockout tissues. Further, the method is easily adapted to high throughput as it is cell-free, conducted at room temperature and is relatively brief (1 hour reaction time period versus previous period of 2 days).

While the instant disclosure sets forth an effective working embodiment of the invention, those skilled in the art are able to optimize the assay in a variety of ways, all of which are encompassed by the invention. For example, charcoal is very efficient (>98%) at removing the unused portion of the stearoyl-CoA but has the disadvantage of being messy and under some conditions difficult to pipette. It may not be necessary to use charcoal if the stearoyl-CoA complex sufficiently aggregates when acidified and spun under moderate g force. This can be tested by measuring the signal/noise ratio with and without charcoal following a desaturation reaction. There are also other reagents that would efficiently sediment stearoyl-CoA to separate it from the ³H-water product.

The following assays are also suitable for measuring SCD1 biological activity in the presence of potential agents. These assays are also valuable as secondary screens to further select SCD1 specific inhibitors from a library of potential therapeutic agents.

Cellular based desaturation assays can be used to track SCD1 activity levels. By tracking the conversion of stearate to oleate in cells (3T3L1 adipocytes are known to have high SCD1 expression and readily take up stearate when complexed to BSA) one can evaluate compounds found to be inhibitory in the primary screen for additional qualities or characteristics such as whether they are cell permeable, lethal to cells, and/or competent to inhibit SCD1 activity in cells. This cellular based assay may employ a recombinant cell line containing a SCD1. The recombinant gene is optionally under control of an inducible promoter and the cell line preferably over-expresses SCD1 protein.

Other assays for tracking other SCD isoforms can be developed. For example, rat and mouse SCD2 is expressed in brain. A microsome preparation can be made from the brain as previously done for SCD1 from liver. The object may be to find compounds that would be specific to SCD1. This screen would compare the inhibitory effect of the compound for SCD1 versus SCD2.

Although unlikely, it is possible that a compound “hit” in the SCD1 assay may result from stimulation of an enzyme present in the microsome preparation that competitively utilizes stearoyl-CoA at the expense of that available for SCD1-dependent desaturation. This would mistakenly be interpreted as SCD1 inhibition. One possibility to examine this problem would be incorporation into phospholipids of the unsaturated lipid (stearate). By determining effects of the compounds on stimulation of stearate incorporation into lipids researchers are able to evaluate this possibility.

Cell based assays may be preferred, for they leave the SCD1 gene in its native format. Particularly promising for SCD1 analysis in these types of assays are fluorescence polarization assays. The extent to which light remains polarized depends on the degree to which the tag has rotated in the time interval between excitation and emission. Since the measurement is sensitive to the tumbling rate of molecules, it can be used to measure changes in membrane fluidity characteristics that are induced by SCD1 activity—namely the delta-9 desaturation activity of the cell. An alternate assay for SCD1 involves a FRET assay. FRET assays measure fluorescence resonance energy transfer which occurs between a fluorescent molecule donor and an acceptor, or quencher. Such an assay may be suitable to measure changes in membrane fluidity or temperature sensitivity characteristics induced by SCD1 biological activity.

The screening assays of the invention may be conducted using high throughput robotic systems. In the future, preferred assays may include chip devices developed by, among others, Caliper, Inc., ACLARA BioSciences, Cellomics, Inc., Aurora Biosciences Inc., and others.

In other embodiments of an SCD1 assay, SCD1 biological activity can also be measured through a cholesterol efflux assay that measures the ability of cells to transfer cholesterol to an extracellular acceptor molecule and is dependent on ABCA1 function. A standard cholesterol efflux assay is set out in Marcil et al., Arterioscler. Thromb. Vasco Bioi. 19:159-169, 1999, incorporated herein by reference in its entirety.

Preferred assays are readily adapted to the format used for drug screening, which may consist of a multi-well (e.g., 96-well, 384 well or 1,536 well or greater) format. Modification of the assay to optimize it for drug screening would include scaling down and streamlining the procedure, modifying the labeling method, altering the incubation time, and changing the method of calculating SCD1 biological activity and so on. In all these cases, the SCD1 biological activity assay remains conceptually the same, though experimental modifications may be made.

Another preferred cell based assay is a cell viability assay for the isolation of SCD1 inhibitors. Overexpression of SCD1 decreases cell viability. This phenotype can be exploited to identify inhibitory compounds. This cytotoxicity may be due to alteration of the fatty acid composition of the plasma membrane. In a preferred embodiment, the human SCD1 cDNA would be placed under the control of an inducible promoter, such as the Tet-On Tet-Off inducible gene expression system (Clontech). This system involves making a double stable cell line. The first transfection introduces a regulator plasmid and the second would introduce the inducible SCD1 expression construct. The chromosomal integration of both constructs into the host genome would be favored by placing the transfected cells under selective pressure in the presence of the appropriate antibiotic. Once the double stable cell line was established, SCD1 expression would be induced using the tetracycline or a tetracycline derivative (e.g., Doxycycline). Once SCD1 expression had been induced, the cells would be exposed to a library of chemical compounds for high throughput screen of potential inhibitors. After a defined time period, cell viability would then be measured by means of a fluorescent dye or other approach (e.g., turbidity of the tissue culture media). Those cells exposed to compounds that act to inhibit SCD1 activity would show increased viability, above background survival. Thus, such an assay would be a positive selection for inhibitors of SCD1 activity based on inducible SCD1 expression and measurement of cell viability.

An alternative approach is to assay SCD activity is to measure the interference of the desaturase system. As described earlier, the desaturase system has three major proteins: cytochrome b₅, NADH (P)-cytochrome b₅ reductase, and terminal cyanide-sensitive desaturase. Terminal cyanide-sensitive desaturase is the product of the SCD gene. SCD activity depends upon the formation of a stable complex between the three aforementioned components. Thus, any agent that interferes with the formation of this complex or any agent that interferes with the proper function of any of the three components of the complex would effectively inhibit SCD activity.

Another type of modulator of SCD1 activity involves a 33 amino acid destabilization domain located at the amino terminal end of the pre-SCD1 protein (Mziaut et al., PNAS 2000, 97: p 8883-8888). It is possible that this domain may be cleaved from the SCD1 protein by an as yet unknown protease. This putative proteolytic activity would therefore act to increase the stability and half-life of SCD1. Inhibition of the putative protease, on the other hand, would cause a decrease in the stability and half life of SCD1. Compounds which block or modulate removal of the destabilization domain therefore will lead to reductions in SCD1 protein levels in a cell. Therefore, in certain embodiments of the invention, a screening assay will employ a measure of protease activity to identify modulators of SCD1 protease activity. The first step is to identify the specific protease which is responsible for cleavage of SCD1. This protease can then be integrated into a screening assay. Classical protease assays often rely on splicing a protease cleavage site (i.e., a peptide containing the cleavable sequence pertaining to the protease in question) to a protein, which is deactivated upon cleavage. A tetracycline efflux protein may be used for this purpose. A chimera containing the inserted sequence is expressed in E. coli. When the protein is cleaved, tetracycline resistance is lost to the bacterium. In vitro assays have been developed in which a peptide containing an appropriate cleavage site is immobilized at one end on a solid phase. The other end is labeled with a radioisotope, fluorophore, or other tags. Enzyme-mediated loss of signal from the solid phase parallels protease activity. These techniques can be used both to identify the protease responsible for generating the mature SCD1 protein, and also for identifying modulators of this protease for use in decreasing SCD1 levels in a cell.

An SCD1 activity assay may also be carried out as a cell free assay employing a cellular fraction, such as a microsomal fraction, obtained by conventional methods of differential cellular fractionation, most commonly by ultracentrifugation methods.

When any agent is tested in animals including humans, SCD biological activity can be measured indirectly by the ratio of 18:1 to 18:0 fatty acids in the total plasma lipid fraction.

3. SCD1-Containing Genetic Constructs and Recombinant Cells that can be used for SCD1 Production and Screening Assays

In certain embodiments, screening protocols to develop agents to practice the present invention might contemplate use of a SCD1 gene or protein in genetic constructs or recombinant cells or cell lines. SCD1 recombinant cells and cell lines may be generated using techniques known in the art, and those more specifically set out below.

Genetic constructs (e.g., vectors) which contain a SCD1 gene can be generated and introduced into host cells, especially where such cells result in a cell line that can be used for assay of SCD1 activity, and production of SCD1 polypeptides by recombinant techniques.

The host cell can be a higher eukaryotic cell, such as a mammalian cell or an insect cell (e.g., SF9 cells from Spodoptera frugiperda), or a lower eukaryotic cell, such as a yeast cell, or the host cell can be a prokaryotic cell, such as a bacterial cell. The selection of an appropriate host is deemed to be within the knowledge of those skilled in the art based on the teachings herein. Host cells are genetically engineered (transduced or transformed or transfected) with the vectors which may be, for example, a cloning vector or an expression vector. The engineered host cells are cultured in conventional nutrient media modified as appropriate for activating promoters, selecting transformants or amplifying the SCD1 gene. The culture conditions, such as temperature, pH and the like, are those previously used with the host cell selected for expression, and will be apparent to a skilled artisan.

It is well within the knowledge and skill of a skilled artisan to construct a genetic construct or vector containing a SCD1 gene that can be used to express SCD1 at the mRNA or protein level in a cell or cell-free system. Such constructs or vectors may include chromosomal, nonchromosomal and synthetic DNA sequences, e.g., derivatives of SV40, bacterial plasmids, phage DNA, baculovirus, yeast plasmids, vectors derived from combinations of plasmids and phage DNA, viral DNA such as vaccinia, adenovirus, fowl pox virus, and pseudorabies. Appropriate cloning and expression vectors for use with prokaryotic and eukaryotic hosts are described by Sambrook, et al., Molecular Cloning: A laboratory Manual, Second Edition, Cold Spring Harbor, N.Y., (1989), Wu et al., Methods in Gene Biotechnology (CRC Press, New York, N.Y., 1997), Recombinant Gene Expression Protocols, In Methods in Molecular Biology, Vol. 62, (Tuan, ed., Humana Press, Totowa, N.J., 1997), and Current Protocols in Molecular Biology, (Ausabel et al., Eds.,), John Wiley & Sons, NY (1994-1999), the disclosures of which are hereby incorporated by reference in their entirety. The following vectors are provided by way of example; Bacterial: pQE70, pQE60, pQE-9 (Qiagen), pBS, pD10, phagescript, psiX174, pBluescript SK, pBSKS, pNH8A, pNH16a, pNH18A, pNH46A (Stratagene); pTRC99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may also be used as long as they can express SCD1 under suitable conditions.

The appropriate polynucleotide sequence may be inserted into the vector by a variety of procedures. In general, the polynucleotide sequence is inserted into an appropriate restriction endonuclease site(s) by procedures known in the art. Such procedures and others are deemed to be within the scope of those skilled in the art.

The polynucleotide sequence in an expression vector is operatively linked to an appropriate expression control sequence(s) (promoter) to direct mRNA synthesis. Representative examples of such promoters include bacterial promoters such as lacl, lacZ, T3, T7, gpt, lambda P_{R}, P_{L} and trp, and eukaryotic promoters such as CMV immediate early, HSV thymidine kinase, early and late SV40, LTRs from retrovirus and mouse metallothionein-I. Other promoters known to control expression of genes in prokaryotic or eukaryotic cells or their viruses can also be used. Selection of the appropriate vector and promoter is well within the level of ordinary skill in the art. The expression vector may contain a ribosome binding site for translation initiation and a transcription terminator. The vector may also include appropriate sequences for amplifying expression.

In addition, an expression vector preferably contains one or more selectable marker genes to provide a phenotypic trait for selection of transformed host cells such as dihydrofolate reductase or neomycin resistance for eukaryotic cell culture, or such as tetracycline or ampicillin resistance in E. coli.

Transcription of the DNA encoding a SCD1 protein by eukaryotic cells, especially mammalian cells, most especially human cells, can be increased by inserting an enhancer sequence into the expression vector. Enhancers are cis-acting elements of DNA, usually about from 10 to 300 bp that act on a promoter to increase its transcription. Examples include the SV40 enhancer on the late side of the replication origin bp 100 to 270, a cytomegalovirus early promoter enhancer, the polyoma enhancer on the late side of the replication origin, and adenovirus enhancers.

Optionally, a leader sequence capable of directing secretion of translated protein into the periplasmic space or extracellular medium can be included in the expression vector to facilitate downstream applications of the protein generated. Further, extra nucleotide sequences can be added to a SCD1 coding sequence in the expression vector for producing a SCD 1 fusion protein that includes an N-terminal or C-terminal identification peptide imparting desired characteristics, e.g., stabilization or simplified purification of expressed recombinant product.

A Baculovirus-based expression system is especially useful for expressing SCD1 as disclosed herein. Baculoviruses represent a large family of DNA viruses that infect mostly insects. The prototype is the nuclear polyhedrosis virus (AcMNPV) from Autographa californcia, which infects a number of lepidopteran species. One advantage of the baculovirus system is that recombinant baculoviruses can be produced in vivo. Following co-transfection with transfer plasmid, most progeny tend to be wild type and a good deal of the subsequent processing involves screening. To help identify plaques, special systems are available that utilize deletion mutants. By way of non-limiting example, a recombinant AcMNPV derivative (called BacPAK6) has been reported in the literature that includes target sites for the restriction nuclease Bsu361 upstream of the polyhedrin gene (and within ORF 1629) that encodes a capsid gene (essential for virus viability). Bsf361 does not cut elsewhere in the genome and digestion of the BacPAK6 deletes a portion of the ORF1629, thereby rendering the virus non-viable. Thus, with a protocol involving a system like Bsu361-cut BacPAK6 DNA most of the progeny are non-viable so that the only progeny obtained after co-transfection of transfer plasmid and digested BacPAK6 is the recombinant because the transfer plasmid, containing the exogenous DNA, is inserted at the Bsu361 site thereby rendering the recombinants resistant to the enzyme (see Kitts and Possee, A method for producing baculovirus expression vectors at high frequency, BioTechniques, 14,810-817 (1993)). For general procedures, see King and Possee, The Baculovirus Expression System: A Laboratory Guide, Chapman and Hall, New York (1992) and Recombinant Gene Expression Protocols, in Methods in Molecular Biology, Vol. 62, (Tuan, ed., Humana Press, Totowa, N.J., 1997), at Chapter 19, pp. 235-246.

It is understood that a vector construct comprising a SCD1 promoter sequence operably linked to a reporter gene as disclosed herein can be used to study the effect of potential transcription regulatory proteins, and the effect of compounds that inhibit the effect of those regulatory proteins, on the transcription of SCD1.

Factors that may modulate gene expression include transcription factors such as, but not limited to, retinoid X receptors (RXRs), peroxisomal proliferation-activated receptor (PPAR) transcription factors, the steroid response element binding proteins (SREBP-1 and SREBP-2), REV-ERBα, ADD-1, EBPα, CREB binding protein, P300, HNF 4, RAR, LXR, and RORα, NF-Y, C/EBPalpha, PUFA-RE and related proteins and transcription regulators. Screening assays designed to assess the capacity of test compounds to inhibit the ability of these transcription factors to transcribe SCD1 are contemplated by this invention.

In accordance with the foregoing, following identification of chemical agents having the desired inhibiting activity of SCD1, the present invention also relates to a process for treating an animal, especially a human, who suffers from type 2 diabetes involving inhibiting SCD1 activity in said animal. In a preferred embodiment, said inhibition of SCD1 activity is not accompanied by substantial inhibition of activity of delta-5 desaturase, delta-6 desaturase or fatty acid synthetase. In a specific embodiment, the present invention relates to a process for increasing insulin sensitivity comprising administering to said animal an effective amount of an agent whose activity was first identified by the process of the invention.

In accordance with the foregoing, the present invention also relates to an inhibitor of SCD1 activity and which is useful for increasing insulin sensitivity wherein said activity was first identified by its ability to inhibit SCD1 activity, especially where such inhibition was first detected using a process as disclosed herein according to the present invention. In a preferred embodiment thereof, such inhibiting agent does not substantially inhibit delta-5 desaturase, delta-6 desaturase or fatty acid synthetase.

In accordance with the foregoing, the present invention further relates to a process for increasing insulin sensitivity in an animal, comprising administering to said animal an effective amount of an agent for which such insulin sensitivity increasing activity was identified by a process as disclosed herein according to the invention.

In a preferred embodiments of such process, the inhibiting agent does not substantially inhibit delta-5 desaturase, delta-6 desaturase or fatty acid synthetase.

4. Test Compounds/Inhibitors/Library Sources

In accordance with the foregoing, the present invention also relates to agents, regardless of molecular size or weight, effective in increasing insulin sensitivity, and/or treating or preventing type 2 diabetes, preferably where such agents have the ability to inhibit the activity and/or expression of the SCD1, and most preferably where said agents have been determined to have such activity through at least one of the screening assays disclosed according to the present invention.

Test compounds are generally compiled into libraries of such compounds, and a key object of the screening assays of the invention is to select which compounds are relevant from libraries having hundreds of thousands, or millions of compounds.

Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

Thus, in one aspect the present invention relates to agents capable of inhibiting the activity and/or expression of SCD1, especially where said inhibiting ability was first determined using an assay involving the use of SCD1 protein or a SCD1 gene, or an assay which measures SCD1 activity. As used herein the term “capable of inhibiting” refers to the characteristic of such an agent whereby said agent has the effect of inhibiting the overall biological activity of SCD1, either by decreasing said activity, under suitable conditions of temperature, pressure, pH and the like so as to facilitate such inhibition to a point where it can be detected either qualitatively or quantitatively and wherein such inhibition may occur in either an in vitro or in vivo environment. In addition, while the term “inhibition” used herein to mean a decrease in activity, the term “activity” not to be limited to specific enzymatic activity alone (for example, as measured in units per milligram or some other suitable unit of specific activity) but includes other direct and indirect effects of the protein, including decreases in enzyme activity due not to changes in specific enzyme activity but due to changes of expression of polynucleotides encoding and expressing said SCD1 enzyme. Human SCD1 activity may also be influenced by agents which bind specifically to substrates of hSCD 1. Thus, the term “inhibition” used herein means a decrease in SCD1 activity regardless of the molecular or genetic level of said inhibition, be it an effect on the enzyme per se or an effect on the genes encoding the enzyme or on the RNA, especially mRNA, involved in expression of the genes encoding said enzyme. Thus, modulation by such agents can occur at the level of DNA, RNA or enzyme protein and can be determined either in vivo or ex vivo.

In specific embodiments thereof, said assay is any of the assays disclosed herein according to the invention. In addition, the agent(s) contemplated by the present disclosure includes agents of any size or chemical character, either large or small molecules, including proteins, such as antibodies, nucleic acids, either RNA or DNA, and small chemical structures, such as small organic molecules.

5. Combinatorial and Medicinal Chemistry

Typically, a screening assay, such as a high throughput screening assay, will identify several or even many compounds which modulate the activity of the assay protein. A compound identified by the screening assay may be further modified before it is used in animals as a therapeutic agent. Typically, combinatorial chemistry is performed on the inhibitor, to identify possible variants that have improved absorption, biodistribution, metabolism and/or excretion, or other important aspects. The essential invariant is that the improved compounds share a particular active group or groups which are necessary for the desired inhibition of the target protein. Many combinatorial chemistry and medicinal chemistry techniques are well known in the art. Each one adds or deletes one or more constituent moieties of the compound to generate a modified analog, which analog is again assayed to identify compounds of the invention. Thus, as used in this invention, compounds identified using a SCD1 screening assay of the invention include actual compounds so identified, and any analogs or combinatorial modifications made to a compound which is so identified which are useful for increasing insulin sensitivity.

III. Pharmaceutical Preparations and Dosages

In another aspect the present invention is directed to compositions comprising the polynucleotides, polypeptides or other chemical agents, including therapeutic or prophylactic agents, such as small organic molecules, disclosed herein according to the present invention wherein said polynucleotides, polypeptides or other agents are suspended in a pharmacologically acceptable carrier, which carrier includes any pharmacologically acceptable diluent or excipient. Pharmaceutically acceptable carriers include, but are not limited to, liquids such as water, saline, glycerol and ethanol, and the like. A thorough discussion of pharmaceutically acceptable carriers, diluents, and other excipients is presented in REMINGTON'S PHARMACEUTICAL SCIENCES (Mack Pub. Co., NJ, current edition), which is herein incorporated by reference in its entirety.

The inhibitors utilized above may be delivered to a subject using any of the commonly used delivery systems known in the art, as appropriate for the inhibitor chosen. The preferred delivery systems include intravenous injection or oral delivery, depending on the ability of the selected inhibitor to be adsorbed in the digestive tract. Any other delivery system appropriate for delivery of small molecules, such as skin patches, may also be used as appropriate.

In another aspect the present invention further relates to a process for preventing or treating type 2 diabetes in a patient afflicted therewith comprising administering to said patient a therapeutically or prophylactically effective amount of a composition as disclosed herein.

IV. Diagnosis and Pharmacogenomics

In an additional aspect, the present invention also relates to a process for diagnosing a disease or condition in an animal, such as a human being, suspected of being afflicted therewith, or at risk of becoming afflicted therewith, comprising obtaining a tissue sample from said animal and determining the level of activity of SCD1 in the cells of said tissue sample and comparing said activity to that of an equal amount of the corresponding tissue from an animal not suspected of being afflicted with, or at risk of becoming afflicted with, said disease or condition. In specific embodiments thereof, said disease or condition includes, but is not limited to, type 2 diabetes.

In an additional aspect, this invention teaches that SCD1 has pharmacogenomic significance. Variants of SCD1 including SNPs (single nucleotide polymorphisms), cSNPs (SNPs in a cDNA coding region), polymorphisms and the like may have dramatic consequences on a subject's response to administration of a prophylactic or therapeutic agent. Certain variants may be more or less responsive to certain agents. In another aspect, any or all therapeutic agents may have greater or lesser deleterious side-effects depending on the SCD1 variant present in the subject.

In a pharmacogenomic application of this invention, an assay is provided for identifying cSNPs (coding region small nucleotide polymorph isms) in SCD1 of an individual which are correlated with human disease processes or response to medication. Researchers have identified two putative cSNPs of hSCD1 to date: in exon 1, a C/A SNP at nt 259, corresponding to a D/E amino acid change at position 8; and in exon 5, a C/A cSNP at nt 905, corresponding to a L/M amino acid change at position 224 (sequence numbering according to GenBank Accession: AF097514). It is anticipated that these putative cSNPs may be correlated with human disease processes or response to medication of individuals who contain those cSNPs versus a control population. Those skilled in the art are able to determine which disease processes and which responses to medication are so correlated.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

In applying the disclosure, it should be kept clearly in mind that other and different embodiments of the methods disclosed according to the present invention will no doubt suggest themselves to those of skill in the relevant art.

### EXAMPLE 1

Materials and Methods

Animal experiments. SCD1−/− mice were generated as described in Miyazaki, M. et al. (2001) J Nutr. 131, 2260-2268. Pre bred homozygous (SCD1−/−) and wild-type (SCD1+/+) male mice on an SV129 background were used. Mice were maintained on a 12 h dark/light cycle and were fed a normal nonpurified diet (5008 test diet; PMI Nutrition International Inc., Richmond, Ind.). Mice were housed and bred in a pathogen free barrier facility of the Department of Biochemistry, the University of Wisconsin-Madison. The breeding of these animals was in accordance with the protocols approved by the animal care research committee of the University of Wisconsin-Madison. Male SCD1−/− and SCD1+/+ were sacrificed at 12 weeks of age; gastrocnemius and soleus muscles were extracted and used throughout the study. The plasma insulin and glucose levels were determined using kits (Lincoln Res. and Sigma).

Evaluation ofphosphorylation status of insulin signaling cascade proteins. The phosphorylation assays were carried out as described in Dominici, F. P. et al. (2000) J. Endocrinol. 166, 579-590. Muscle samples were homogenized and centrifuged at 100,000×g for 1 h in ice-cold 50 mM HEPES buffer (pH 7.4) containing 150 mM NaCl, 10 mM sodium pyrophosphate, 2 mM Na₃V0₄, 10 mM NaF, 2 mM EDTA, 2 mM phenylmethylsulfonyl fluoride (PMSF), 5 μg/ml leupeptin, 1% NP-40, and 10% glycerol. Supernatants were collected and protein concentration was measured with Bradford protein assay reagent (Bio-Rad) using BSA as standard. Tissue homogenates (1 mg) were then immunoprecipitated with 4 μg of anti IR, IRS-1, IRS-2 or IGF-1Rβ, antibodies (Santa Cruz, Calif.) for 18 h. Immunoprecipitates were washed three times by brief centrifugation and gentle suspension in ice-cold homogenization buffer plus 0.1% SDS and then were subjected to SDS-PAGE on 10% gradient gel. Proteins were transferred and immobilized on immobile P transfer membrane. The membranes were immunoblotted with antiphosphotyrosine antibodies (Upstate Biotechnology, Inc., Lake Placid, N.Y.) and bands were visualized using ECL and quantified by densitometry. To measure IRS-1 or IRS-2 associated p85 subunit of PI 3-kinase, equal amounts of protein (1 mg) were immunoprecipitated with either IRS-1 or IRS-2 and then immunoblotted with antibody specific to αp85 subunit of PI3-kinase (Santa Cruz, Calf.). Akt/PKB serine and threonine phosphorylation was measured using the phospho Ser 473 and Thr 308 antibodies (Cell Signaling Technology, Inc, Beverly, Mass.). Immunoprecipitation and western blotting procedures are the same as described for IR, IRS-1, IRS-2 IGF-1R tyrosine phosphorylations.

PTP-1B and LAR phosphatase expression. Total RNA was isolated from muscle of 12-week old SCD1+/+ and SCD1−/− male mice using Trizol reagent (Invitrogen) and then analyzed by RT-PCR using PTP-1B specific primers. Real-time quantitive PCR was performed with a Cephied Smart Cycler by monitoring the increase in fluorescence due to the binding of SYBER Green to double-stranded DNA (Miyazaki, M. et al. (2002) J. Lipid Res. 43, 2146-2154). The PTB-1B and LAR protein levels were assessed by Immunoblotting using polyclonal antibodies against PTP-1B and LAR (Santa Cruz, Calif.), respectively. The PTP-1B activity was measured using p-nitrophenyl phosphate (pNPP) as substrate (Shimuzu, S. et al. (2002) Endocrinology 143, 4563-4569).

Determination of plasma membrane GLUT4 levels, glucose uptake and glucose oxidation. Muscle plasma membranes were prepared from muscle of SCD1−/− and SCD1+/+ mice and GLUT4 levels were determined as described in Agote, M. et al. (2001) Am. J. PhysioL. 281, El 101E1109. In vivo glucose uptake assay was carried out as described in Dobrzyn, A., and Gorski, J. (2002) Am. J. Physiol. 281, E277-E285. Mice were anesthetized and 0.2 μCi of 2-deoxy-D-[1-¹⁴] glucose (55 mCi/mmol) and 0.8 μCi of [1-³H] mannitol (20 Ci/mmol) per 20 g body wt were administered into the tail vein of SCD1+/+ and SCD 1−/− mice. [1-³H] mannitol was used to measure the extracellular space. The blood and the muscles were isolated after 25 min. The samples were digested with 1 M KOH followed by neutralization with 1 M HCl. The scintillation cocktail was added and radioactivity was counted in a liquid scintillation counter. The 2-deoxyglucose (2-DG) uptake was calculated as the difference between the total muscle radioactivity and the radioactivity of the muscle extracellular space. In vitro glucose uptake assay was carried out as described in Turinsky, J. et al. (1996) Biochem. J. 313, 199-206. The media used for muscle incubation were equilibrated with 95% O₂/5% CO₂ before use and all incubations were carried out at 37° C. under an atmosphere of 95% O₂/5% CO₂. After incubation the muscle and aliquots of incubation medium were digested in 1 M KOH and the cellular uptake of radioactive 2-DG was determined as described above. Glucose oxidation was determined in thin slices (20-30 mg) of gastrocnemius muscle as described in Baque, S. et al. (2001) Am. J. Physiol. 281, E335-E340.

Measurement of glycogen. Glycogen content in muscle was measured as described in Lo, S. et al. (1970) J. Appl Physiol. 28, 234-236. To determine glycogen accumulation, sections of gastrocnemius muscle of 2 to 3 mm in diameter were fixed in buffered 10% formalin and following dehydration, were embedded in Paraplast. Sections (4-6 μm thick) were cut, dewaxed, and rehydrated and standard Periodic acid-Schiff (PAS) reaction was performed. Glycogen synthase and phosphorylase activities were assayed in gastrocnemius muscle homogenates as described in Golden, S. et al. (1977) Anal. Biochem. 77, 436-445.

Results

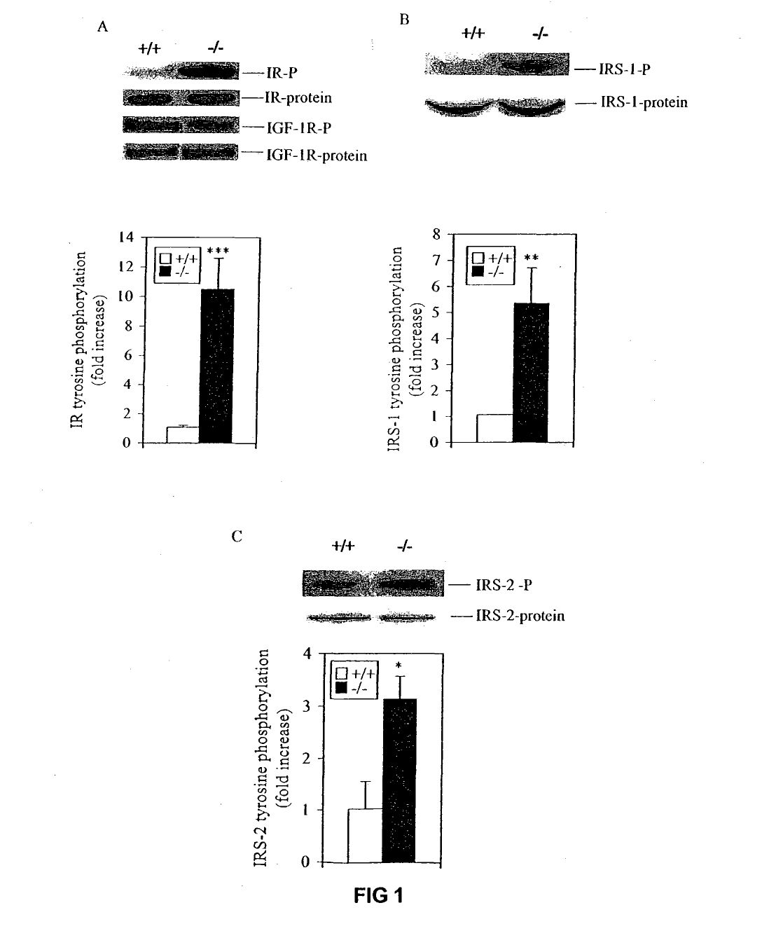

Increased basal tyrosinephosphorylation of IR and IRSs in SCD1−/− mice. We first measured the plasma glucose and insulin levels of SCD1−/− and SCD1+/+ mice. The non-fasting plasma insulin levels were lower in the SCD1−/− mice than the SCD1+/+ mice (SCD1−/−; 0.645±0.053 ng/ml; SCD1+/+; 1.245±0.106 ng/ml, n=6, P<0.005). The glucose levels also tended to be lower in the SCD1−/− mice compared to the controls (SCD1−/−88.8±1.96; SCD1+/+111.7±7.4, n=6). To assess the phosphorylation status of the insulin receptor, immunoprecipitated insulin receptor, was subjected to Western blotting with anti-phoshotyrosine antibodies (FIG. 1A). Densitometric analysis revealed that in spite of the lower levels of plasma insulin, the basal insulin receptor tyrosine phosphorylation was 10-fold higher (P<0.0005) in the muscle of the SCD1−/− mice compared to the wild type mice. In order to determine whether the phosphorylation of the proximal elements of the insulin-signaling cascade were also increased in the basal state, we assessed the degree of IRS-1 and IRS-2 tyrosine phosphorylation as well as the protein levels. IRS-1 tyrosine phosphorylation was 5-fold higher (P<0.005) in the muscle of SCD1−/− mice compared to the wild type mice (FIG. 1B). IRS-2 tyrosine phosphorylation was 3-fold higher (P<0.01) in the SCD1−/− mice than controls (FIG. 1C). There was no significant difference in the IR and IRS-2 protein levels between the two groups of mice. The IRS-I protein levels were 1.5-fold higher (P<0.05) in the SCD1−/− mice. To determine whether the increased phosphorylation is specific to the insulin signaling pathway, we examined the phosphorylation status of IGF-1 receptor which upon tyrosine phosphorylation is also known to regulate signaling via the shc/mitogen-activated protein kinase leading to metabolic changes in muscle (Chow, et al. (1998) J. Biol Chem. 273, 4672-4680; Liu, et al. (1993) Cell. 75, 59-72; Di Cola, et al. (1997) J. Clin. Invest. 99, 2538-2544). As shown in FIG. 1A the tyrosine phosphorylation of the IGF-1 receptor and protein levels were similar between SCD1+/+ and SCD1−/− mice. Thus, increased IR, IRS-1 and IRS-2 tyrosine phosphorylation is consistent with specific to the insulin signaling pathway in the SCD1−/− mice.

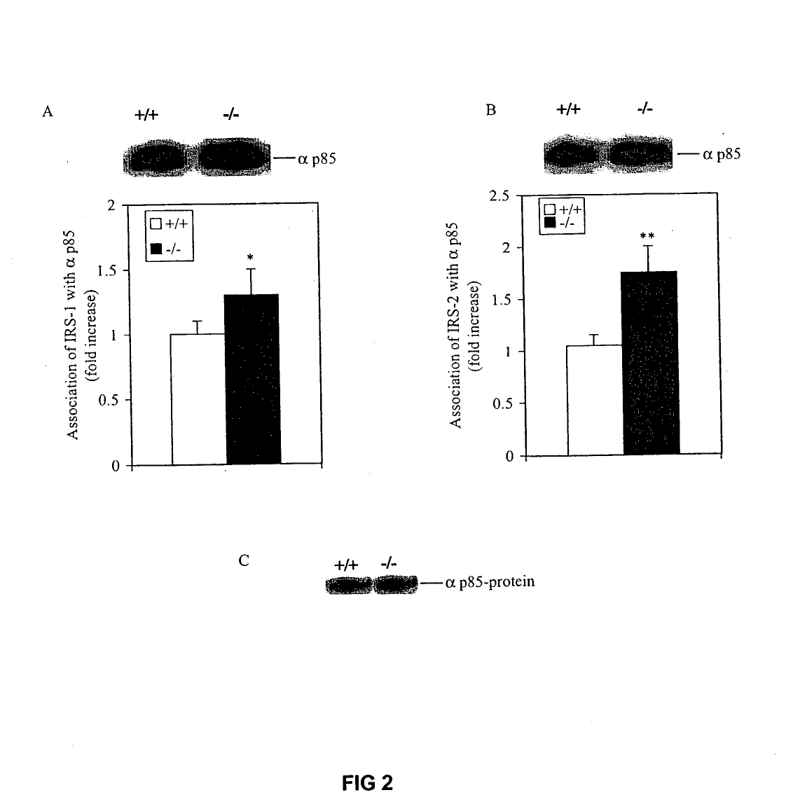

Increased αp85 association with the IRSs in SCD1−/− mice. It is known that when tyrosine residues of insulin receptor substrates are phosphorylated, they associate with p85subunit of PI 3-kinase resulting in its activation (Withers, D. J. et al. (1998) Nature. 391, 900-904) and involvement in insulin signal transduction. The association of p85 subunit of PI-3-kinase with IRS-1 (FIG. 2A) and IRS-2 (FIG. 2B) was 1.3-(P<0.05,) and 1.7-fold (P<0.01), respectively, higher in the SCD1−/− mice compared to SCD1+/+ mice. There was no change in the levels of p85 protein (FIG. 2C).

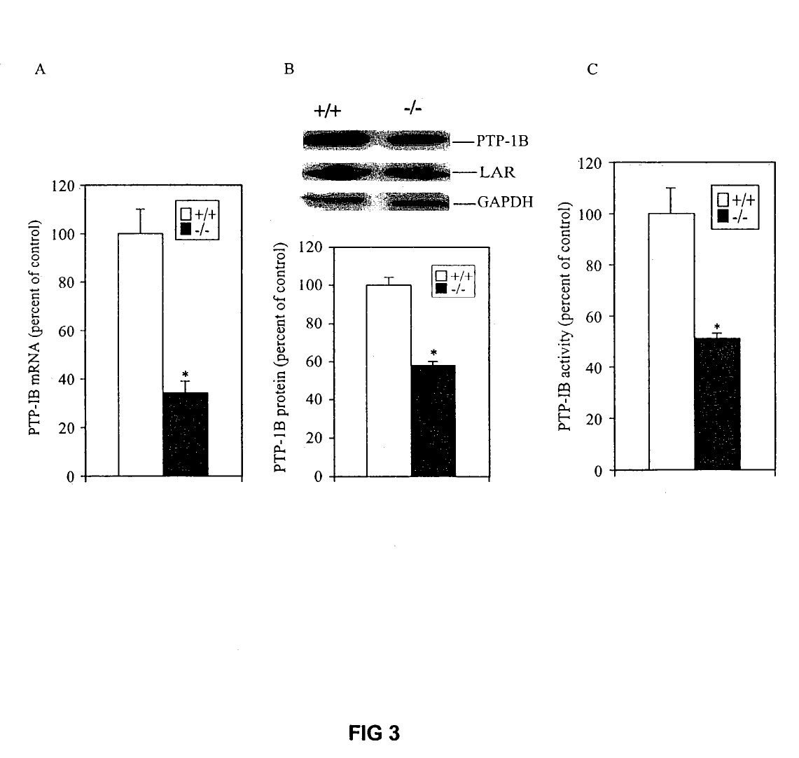

Reduced PTP-1B expression in SCD1−/− mice. Protein-tyrosine phosphatases, particularly PTP-1B, play an important role in regulating the phosphorylation status of proteins involved in insulin signaling. To investigate the possible role of PTP-1B in signal transduction, experiments were conducted to measure the expression, protein mass and activity of PTP-1B in muscle of SCD1−/− and SCD1+/+ mice. RT-PCR analysis using total RNA prepared from muscle shows more than 66% reduction (P<0.001) in PTP-1B mRNA expression in SCD1−/− compared to CD1+/+ mice (FIG. 3A). The protein mass was analyzed using a specific anti-PTP-1B polyclonal antibody. FIG. 3B shows that the PTP-1B protein levels were 42% lower (P<0.001) in SCD1−/− compared to SCD1+/+ mice. Consistent with reduction in protein mass, the PTP-1B activity in muscle of SCD1−/− was reduced by 49% (P<0.001) compared with that in muscle of control mice (FIG. 3C). To determine whether the downregulation of PTP-1B is specific to the insulin signaling pathway in the SCD1−/− mice, we examined the protein levels of the leukocyte antigen related (LAR) protein phosphatase a protein tyrosine phosphatase that has a wide tissue distribution and implicated in negatively regulating the insulin receptor signaling (Mooney, et al. (2003) Curr. Top.Med. Chem. 3, 809-17). As shown in FIG. 3A the protein levels of LAR were similar between SCD1+/+ and SCD1−/− mice.

Without intending to be limited by theory, we propose from the results here that downregulation of the PTP-1B expression and activity is responsible for the sustained insulin receptor autophosphorylation despite reduced level of plasma insulin in the SCD−/− mice.

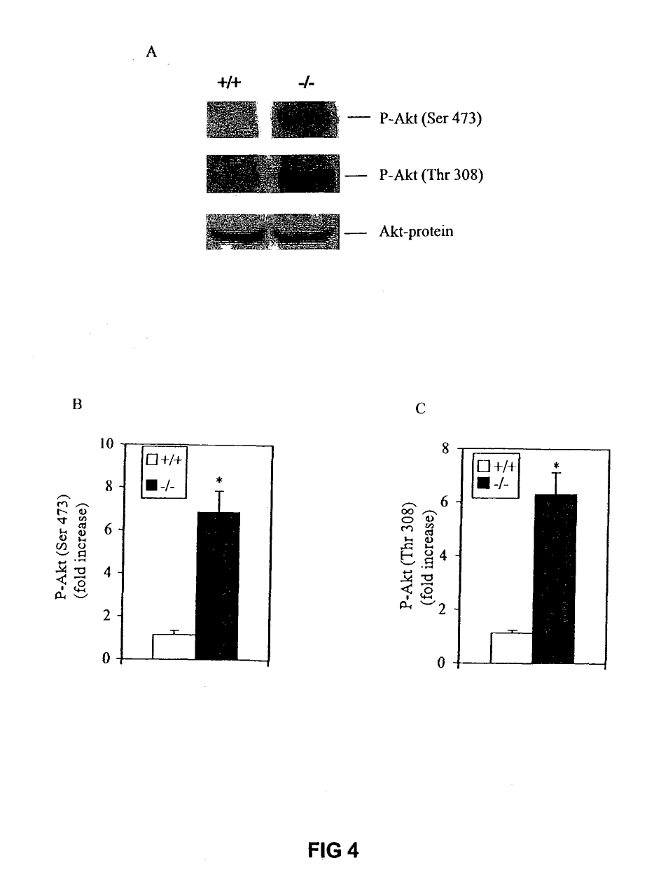

Increased phosphorylation of Akt/PKB in the SCD1−/− mice. In order to investigate insulin signaling status downstream of PI 3-kinase, we examined the phosphorylation status of serine 473 and threonine 308 of Akt/PKB, a key serine/threonine kinase, which mediates many metabolic effects of insulin including activation of GLUT4 translocation to the plasma membrane (Holman, et al. (1997) Diabetologia. 40, 991-1003; Kohn, et al. (1995) EMBO J. 14, 4288-4295). The immunoblot analysis in FIG. 4A and the densitometric analysis show that serine 473 (FIG. 4B) and threonine 308 (FIG. 4C) phosphorylation was 6-fold (P<0.005) and 5-fold higher (P<0.005), respectively, in SCD1−/− mice compared to SCD1+/+ mice indicating that phosphorylation of Akt/PKB were significantly increased in the SCD1−/− mice. Immunoblotting for Akt mass (FIG. 4A) did not show significant differences between the SCD1−/− and SCD1+/+ mice.

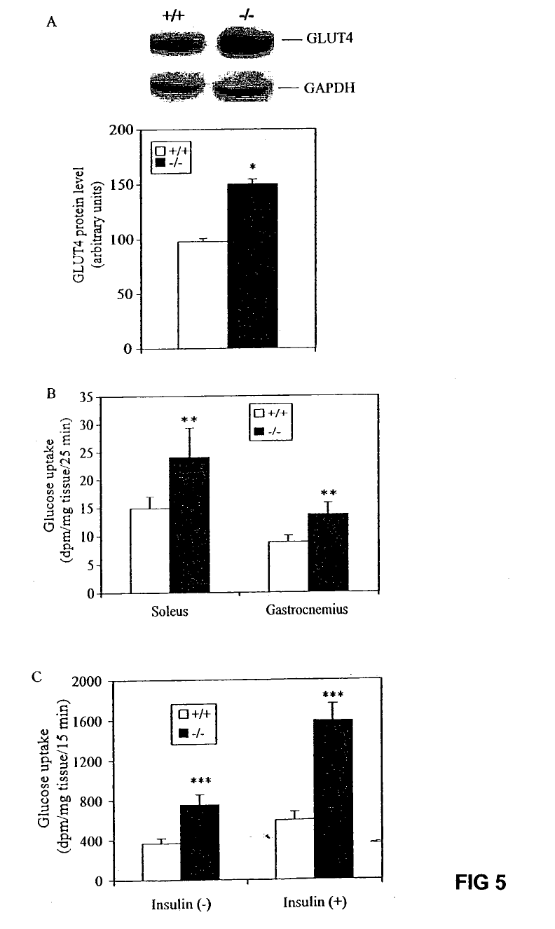

Increased levels of GLUT4 in plasma membrane of SCD1−/− mice. The elevation of the insulin signaling components would be expected to lead to increased uptake of glucose into cells by the glucose transporter GLUT4. We determined by Western blotting the changes in the levels of GLUT4 in the plasma membranes isolated from muscle of SCD1−/− and SCD1+/+ mice (FIG. 5A). Densitometric analysis shows that the GLUT4 levels in the plasma membrane of SCD1−/− mice are 1.5-fold higher (P<0.05) compared to SCD1+/+ mice. The GAPDH antibody was used as control for loading and as shown the GAPDH levels were not altered in the plasma membranes of the SCD1−/− and SCD1+/+ mice. We then measured in vivo deoxyglucose uptake in muscle to determine whether the increase in GLUT4 levels in the plasma membrane of the SCD1−/− mice results in increased glucose uptake. Radioactive deoxyglucose was injected intravenously and its distribution in muscle of the SCD1−/− and SCD1+/+ mice was determined. Radioactive mannitol was used as an internal control. There was a 1.5-fold (P<0.01) and 1.7-fold (P<0.01) increase in 2-deoxyglucose content in the gastrocnemius and soleus muscles respectively, of SCD1−/− compared to the SCD1+/+ mice (FIG. 5B). In order to determine whether muscle from SCD1−/− mice demonstrated increased insulin responsiveness we performed insulin-stimulated glucose uptake experiments in isolated soleus muscle of both SCD1 −/− and SCD1+/+ mice. As shown in FIG. 5C insulin-mediated glucose uptake was 2.1-fold higher (P<0.001) in the soleus muscle from SCD1−/− compared to a 1.6-fold (P<0.001) in the SCD1+/+ mice (FIG. 5C). Thus, soleus muscle from SCD1−/− mice demonstrated a pronounced elevation of the effects of insulin on glucose uptake.

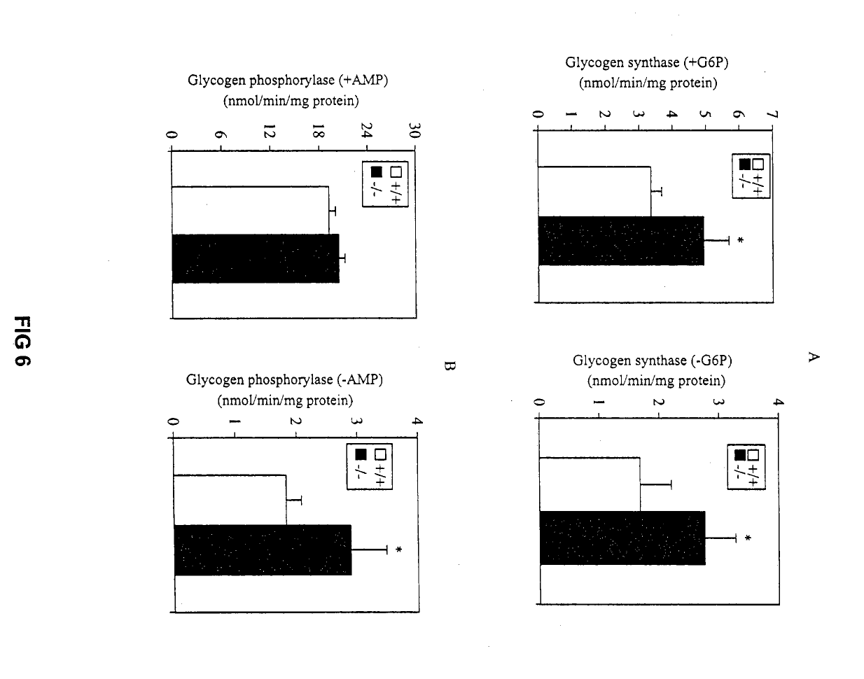

Increased glycogen synthesis and turnover in SCD1−/− mice. To determine whether increased glucose uptake leads to increased glycogen synthesis, we measured the activities of two key enzymes in glycogen metabolism: glycogen synthase and glycogen phosphorylase. Both the total and active forms of glycogen synthase were 1.5-fold (P<0.05) and 1.6-fold higher (P<0.05) respectively, in the muscle of SCD1−/− mice (FIG. 6A). Total glycogen phosphorylase activity was similar between the SCD1−/− mice and wildtype mice but the activity of the active form of glycogen phosphorylase as measured in the absence of AMP was 1.5-fold higher (P<0.05) in SCD1−/− mice (FIG. 6B). The glucose oxidation was similar between the two groups of mice (SCD1+/+, 0.85±0.9 vs SCD1−/−, 0.89±0.11 mmol/h/g tissue) despite increased glycogen synthesis and turnover in the SCD 1−/− mice.

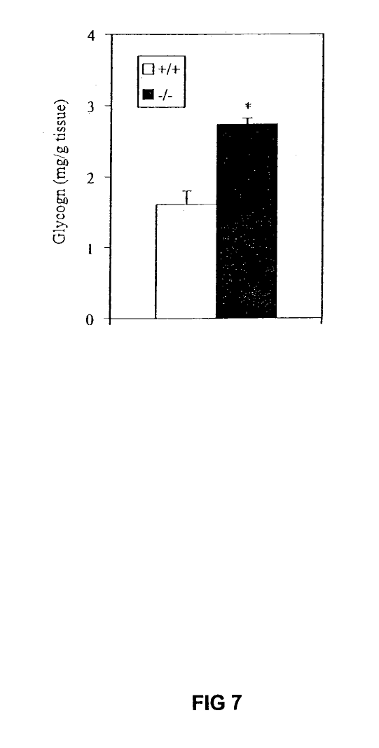

To determine whether increased glycogen synthesis resulted in net glycogen accumulation we measured glycogen content in the muscle of SCD1−/− and SCD1+/+ mice. Chemical determination of glycogen showed 1.8-fold higher (P<0.001) glycogen content in muscle of SCD1−/− mice (FIG. 7). The increased glycogen content was confirmed by light microscopy examination that shows that the muscle of SCD1−/− has more red granules with Periodic Acid-Schiff (PAS) staining than SCD1+/+ mice.

### Example 2

Materials and Methods

Animals and Diets. SCD1−/− mice in SV129 background were generated and genotyped as described in Miyazaki, M. et al. (2001) J. Nutr. 131, 2260-2268. The wild-type (SCD1+/+), heterozygous (SCD1+/−) and homozygous (SCD1−/−) mice are housed and bred in a pathogen-free barrier facility of the Department of Biochemistry (University of Wisconsin, Madison) operating at room temperature in a 12-h light/12-h dark cycle. The breeding of these animals was in accordance with the protocols approved by the animal care research committee of the University of Wisconsin. At 3 weeks of age, the mice were fed ad libitum a standard laboratory chow diet or a high-fat diet for 23 weeks. The high-fat diet contains 195 g/kg casein, 3 g/kg DL-methionine, 377 g/kg sucrose, 150 g/kg corn starch, 153 g/kg anhydrous milkfat, 10 g/kg corn oil, 1.5 g/kg cholesterol, 60.067 g/kg cellulose, 35 g/kg mineral mix AIN-76 (170915), 4 g/kg calcium carbonate, 10 g/kg vitamin mix Teklad (40060), 1.2 g/kg choline bitartrate, and 0.033 g/kg ethoxyquin (antioxidant). The weight of each mouse within each group was measured weekly; the data are presented as means ±SD (n=8, P<0.001). The glucose tolerance and insulin tolerance were determined as described in Stoehr, J. P. et al. (2000) Diabetes 49, 1946-1954.

Measurement of Oxygen Consumption. Gender matched SCD1−/− and wild-type littermates were investigated in indirect calorimeters as described in Lo, H. C. et al. (1997) Am. J. Clin. Nutr. 65, 1384-1390. Oxygen consumption rate (VO₂) and CO₂ production rate (VCO₂) were continuously assayed over 4 consecutive 23-h periods, including 12 h dark (1800-0600) and 11 h light (0600-1700).

Gene Expression Analysis. RNA was isolated from livers of 10 individual 6-week-old female mice by using a standard method described in Bernlohr, D. A. et al. (1985) J. Biol. Chem. 260, 5563-5567. Mouse genome U74A arrays were used to monitor the expression level of approximately 12,000 genes and expressed sequence tags (Affymetrix). Genes differentially expressed were identified by comparing expression levels in SCD 1−/− and wild-type mice (Newton, M. A. et al. (2001) J. Comput. Biol. 37, 37-52; Li, C. & Wong, W. H. (2001) Proc. Natl. Acad. Sci. USA 98, 31-36). For Northern blot analysis, 20 μg of total liver RNA was separated on an 0.8% agarose/formaldehyde gel, transferred onto nylon membrane, and hybridized with cDNA probes for the corresponding genes.

Results

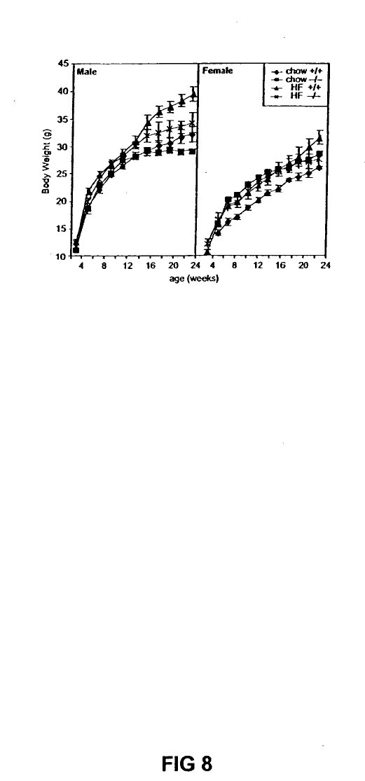

Reduced Body Weight in SCD1−/− Mice Fed a High-Fat Diet. Although the growth curves of male SCD1−/− mice were similar to those of wild-type siblings on chow diet, a high-fat diet revealed large differences in weight gain in both males (34.2 g vs. 39.5 g, P<0.01, FIG. 8) and females (27.7 g vs. 31.9 g, P<0.05).

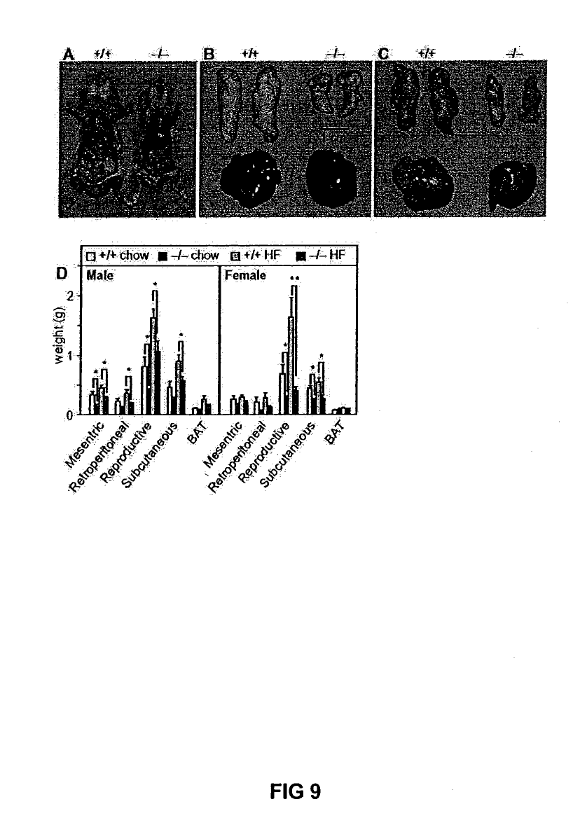

Reduced Body Fat Mass in SCD1−/− Mice. On average, the SCD1−/− mice consumed 25% more food than wild-type mice (4.1 g/day vs. 5.6 g/day; n=9, P<0.05). Nonetheless, they were leaner and accumulated less fat in their adipose tissue (FIG. 9A). The epididymal fat pad mass was markedly reduced in male SCD1 −/− relative to wild-type mice fed a chow diet (0.4±0.1 mg vs. 0.8±0.2; n=9, P<0.05; FIG. 9B) and a high-fat diet (1.0±0.2 mg vs. 1.6±0.2, n=12, P<0.05; FIG. 9C). The livers of the wild-type and SCD1−/− mice were grossly normal and of similar mass. In contrast, on a high-fat diet, the livers of the wild-type mice were lighter in color than those of the mutant mice (FIG. 9C), indicating hepatic steatosis. The masses of white adipose depots in SCD1−/− mice were globally reduced in mice on either the chow or the high-fat diet (FIG. 9D). The masses of other tissues, including brown adipose tissue, were not significantly altered. Thus, SCD1−/− mice were resistant to diet-induced weight gain and fat accumulation, despite increased food intake.

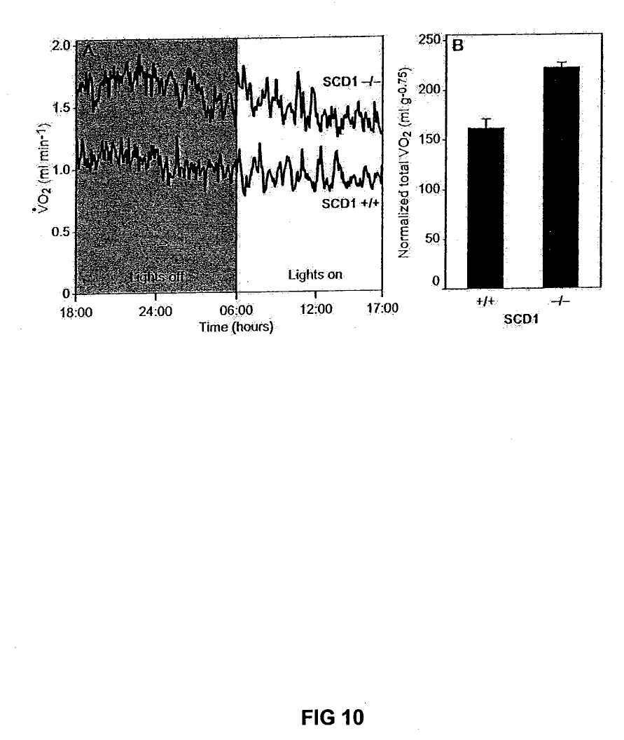

Increased Oxygen Consumption in SCD1/Mice. We carried out indirect calorimetry to investigate whether the resistance to weight gain is caused by increased energy expenditure. The SCD1−/− mice exhibited consistently higher rates of oxygen consumption (had higher metabolic rates) than their wild-type littermates throughout the day and night (FIG. 10A). After adjusting for allometric scaling and gender, the effect of the knockout allele was highly significant (P=0.00019, multiple ANOVA, FIG. 10B).

Because the increase in O₂ consumption occurred during the fasting phase (daytime) as well as during the feeding phase, the animals are more active in oxidizing fat. Although ketone bodies were undetectable in plasma from either strain during postprandial conditions, β-hydroxybutyrate levels were much higher in the SCD1−/− mice after a 4-h fast (4.4±0.6 mg/dl vs. 1.1±0.7 mg/dl; P<0.001), indicating a higher rate of β-oxidation in knockout mice. A similar but less dramatic difference was seen in females. These differences were also observed in mice on high-fat diet.

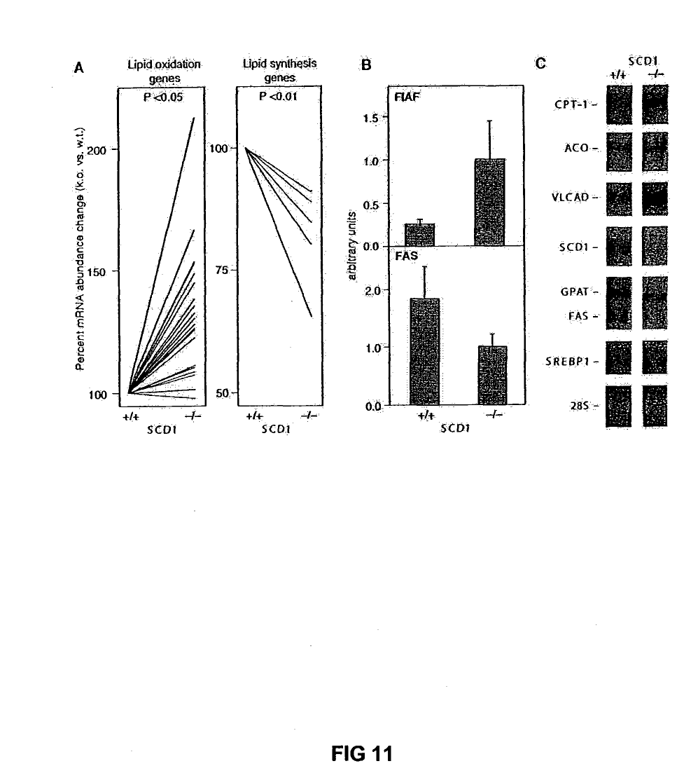

Increased Expression of Genes Involved in Fatty Acid Oxidation in SCD1−/− Mice. We used DNA microarrays to identify genes whose expression was altered in the livers of SCD1−/− mice. We identified 200 mRNAs that were significantly different between the livers of SCD1−/− and wild-type mice. The most striking pattern was seen in genes involved in lipogenesis and fatty acid β-oxidation. Lipid oxidation genes were up-regulated, whereas lipid synthesis genes were down-regulated in the SCD1−/− mice (FIG. 11A). Using the same RNA samples, the microarray data were verified with quantitative reverse-transcription-PCR using DNA primers that were designed for selected genes that showed differential expression (Imanaka, T. et al. (2000) Cell. Biochem. Biophys. 32, 131-138). The results showed that the PPAR-target gene Fasting-Induced Adipocyte Factor (FIAF) was up-regulated in SCD1−/− mice (P<0.05; FIG. 11B), whereas fatty acid synthase (FAS) was down-regulated (P<0.01).

Northern blot analysis also supports changes in fatty acid oxidation and lipid biosynthesis. Probes for acyl-CoA oxidase (ACO), very long chain acyl-CoA dehydrogenase (VLCAD), and carnitine palmitoyltransferase-1 (CPT-1) indicate increases in β-oxidation (Kersten, S. et al. (1999) J. Clin. Invest. 103, 1489-1498; Kersten, S. et al. (2000) J. Biol. Chem. 275, 28488-28493), whereas probes for SREBP-1, FAS, and glycerol phosphate acyl-CoA transferase (GPAT) point to a decrease in triglyceride biosynthesis (FIG. 11C).

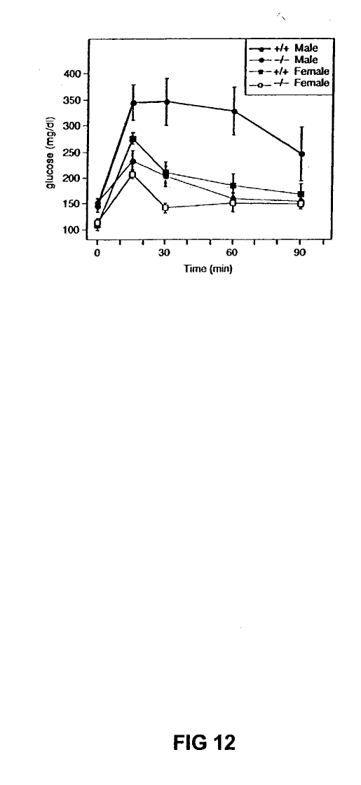

Increased Insulin Sensitivity in SCD1−/− Mice. Reduced adipose tissue mass could either elicit insulin resistance or insulin sensitivity as demonstrated in several animal models (Kersten, S. et al. (2000) J. Biol Chem. 275, 28488-28493). Fasting insulin levels were lower in the male SCD1−/− on chow diet (1.3±0.3 ng/dl; n=7) compared with wild-type mice (2.5±0.9 ng/ml; n=7). On a high-fat diet, insulin levels were similar between the two groups. Fasting glucose levels were similar between the SCD1−/− and wild-type mice. However, male and female SCD1−/− mice showed improved glucose tolerance compared with wild type (FIG. 12, P<0.05). Thirty minutes after a glucose load, both male and female SCD1−/− mice tended to have lower fasting glucose levels (males: wild type, 345±44 mg/dl; SCD1−/− mice, 202±20, n=8; females: wild type, 209±20; SCD1−/− mice, 141±9, n 32 5). In addition, we found that the glucose lowering effect of insulin was greater in the SCD1−/− mice than wild-type mice. These data indicate that SCD 1−/− mice have increased insulin sensitivity along with their loss of adiposity.

We claim:

1. A method of increasing insulin sensitivity in a human or non-human subject, the method comprising the step of:
reducing stearoyl-CoA desaturase 1 (SCD1) activity in the human or non-human subject sufficiently to increase insulin sensitivity.

2. The method of claim 1, wherein reducing SCD1 activity is accomplished by reducing SCD1 protein level.

3. The method of claim 2, wherein reducing SCD 1 protein level is accomplished by inhibiting the transcription of a SCD1 gene.

4. The method of claim 3, wherein inhibiting the transcription of the SCD1 gene is accomplished by administering an agent selected from the group consisting of a thiazoladinedione compound and a polysaturated fatty acid to the subject.

5. The method of claim 4, wherein the thiazoladinedione compound is selected from the group consisting of BRL49653, Pioglitazone, Ciglitazone, Englitazone and Troglitazone.

6. The method of claim 4, wherein the polyunsaturated fatty acid is selected from the group consisting of dodecahexaenoic acid and arachidonic acid.

7. The method of claim 1, wherein the SCD1 protein level is reduced by administering an antisense oligonucleotide for SCD1 into the human or non-human subject.

8. The method of claim 1, wherein reducing SCD1 activity is accomplished by inhibiting the enzymatic activity of SCD1.

9. The method of claim 8, wherein the SCD1 enzymatic activity is inhibited by administering an SCD1 inhibitor into the human or non-human subject.

10. The method of claim 9, wherein the SCD1 inhibitor is an SCD1 antibody.

11. The method of claim 8, wherein the inhibitor inhibits the SCD protein by inhibiting a protein selected from the group consisting of a cytochrome b₅ protein, a NADH-cytochrome b₅ reductase protein, and a terminal cyanide-sensitive desaturase protein.

12. A method for identifying an agent that can increase insulin sensitivity in a human or non-human subject, the method comprising the steps of:
providing a preparation that contains SCD1 activity;
contacting the preparation with a test agent;
measuring SCD1 activity and comparing the activity to that of a control preparation that is not exposed to the test agent, wherein a lower than control activity indicates that the agent can increase insulin sensitivity in a human or non-human subject.

13. A method for identifying an agent that can increase insulin sensitivity in a human or non-human subject, the method comprising the steps of:
administering a test agent to the human or non-human subject; and
determining the effect of the agent on the SCD1 activity in the subject, wherein a reduction in SCD 1 activity caused by the agent indicates that the agent can increase insulin sensitivity in the subject.
